(19) United States
(12) Patent Application Publication
(10) Pub. No.: US 2016/0374557 A1
(43) Pub. Date: Dec. 29, 2016

(54) Portable Health Monitoring System

(71) Applicants: Maryam Bakhshi, Richmond Hill (CA); Ali Farzanfar, Richmond Hill (CA)

(72) Inventors: Maryam Bakhshi, Richmond Hill (CA); Ali Farzanfar, Richmond Hill (CA)

(21) Appl. No.: 15/181,974

(22) Filed: Jun. 14, 2016

Related U.S. Application Data
(60) Provisional application No. 62/183,859, filed on Jun. 24, 2015.

(51) Int. Cl. A61B 5/00; H04B 1/3883; A61B 7/04; A61B 5/024; A61B 5/145; A61B 5/15; A61B 5/021; H04B 1/3827; A61B 5/01

(52) U.S. Cl. CPC A61B 5/0022; H04B 1/3833; H04B 1/385; H04B 1/3883; A61B 5/6887; A61B 7/04; A61B 5/01; A61B 5/14532; A61B 5/150358; A61B 5/0059; A61B 5/150381; A61B 5/150534; A61B 5/681; A61B 5/021; A61B 5/024; H04B 2001/3861; H04W 4/008

(57) ABSTRACT

A portable health monitoring system implements a portable health monitoring apparatus to assess physiological attributes for a user. The physiological attributes include, but are not limited to, internal body temperature, auscultation, and blood glucose levels, as well as ear, nose, and throat irregularities. These physiological attributes are measured through a plurality of health monitoring modules distributed within the portable health monitoring apparatus. The measurements from each of the plurality of health monitoring modules are processed by a central processing unit (CPU) to be transferred through a first wireless transceiver to be received by at least one remote computing device to be assessed professionally.

The current application claims a priority to the U.S. Provisional Patent application Ser. No. 62/183,859 filed on Jun. 24, 2015.

### FIELD OF THE INVENTION

The present invention relates generally to a health monitoring system. More specifically, the present invention relates to a health monitoring system which a user is able to measure health data to be transferred to a health care professional for assessment.

### BACKGROUND OF THE INVENTION

A person's health is very important for remaining fit and active into the later years of life. Regular visits to a doctor have been encouraged for a person to maintain a level of general health. Beyond regular visits, people visit their primary care physician when they are feeling ill. Doctors assess the user's physiological attributes such as body temperature, heart rate and blood pressure, auscultate the heart and lungs, and examine ears nose and throat in order to assess the illness of the user. The doctor then prescribes a treatment based on the assessment to treat the illness. The user is responsible for setting up and arriving to an appointment with the doctor for the assessment of their health.

The present invention is a portable health monitoring system. The present invention allows a user to monitor physiological attributes for the user and transmits the data retrieved from a health monitoring apparatus to a remote computing device. The remote computing device is viewed by the user's physician. The user's physician can assess the data received for any illness related to symptoms shown from differences in physiological attributes and auscultation, as well as, ear, nose, and throat irregularities. The physician is able to prescribe treatment based on their assessment or require the user to come in for a more detailed assessment.

### BRIEF DESCRIPTION OF THE DRAWINGS

FIG. 1 is a perspective view for an embodiment of the portable health monitoring apparatus of the present invention.

FIG. 2 is a lateral view for an embodiment of the portable health monitoring apparatus, normal to the needle housing.

FIG. 3 is a schematic view for an embodiment of the portable health monitoring apparatus along the line 3-3 of FIG. 2.

FIG. 4 is a diagram depicting the data transmission between the portable health monitoring apparatus and the at least one remote computing device.

FIG. 5 is a schematic view for a watch case of a smartwatch, wherein the smartwatch is a remote computing device of the at least one remote computing device.

FIG. 6 is a perspective view of the portable health monitoring apparatus mounted to a remote computing device of the at least one remote computing device.

### DETAIL DESCRIPTIONS OF THE INVENTION

All illustrations of the drawings are for the purpose of describing selected versions of the present invention and are not intended to limit the scope of the present invention.

The present invention is a portable health monitoring system. The present invention allows the user to monitor physiological attributes including, but not limited to, heart rate, blood pressure, blood glucose levels, internal body temperature, and auscultation, as well as ears, nose, and throat irregularities. Data acquired by the present invention is transmitted to a remote computing device for a professional assessment, where the professional is a doctor, nurse, physician or any other medical practitioner.

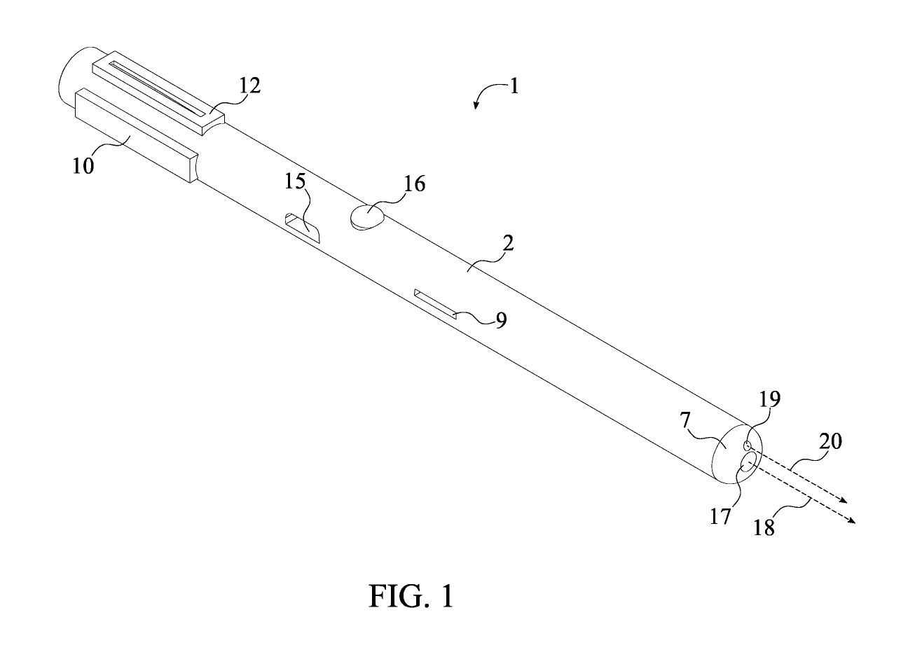

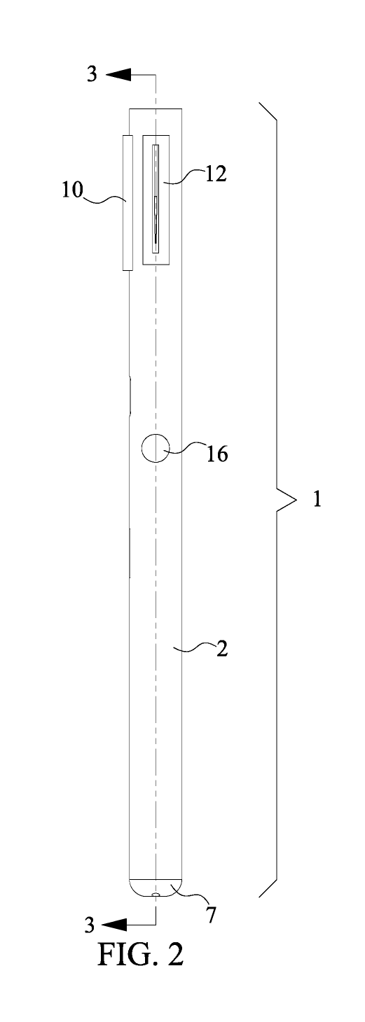

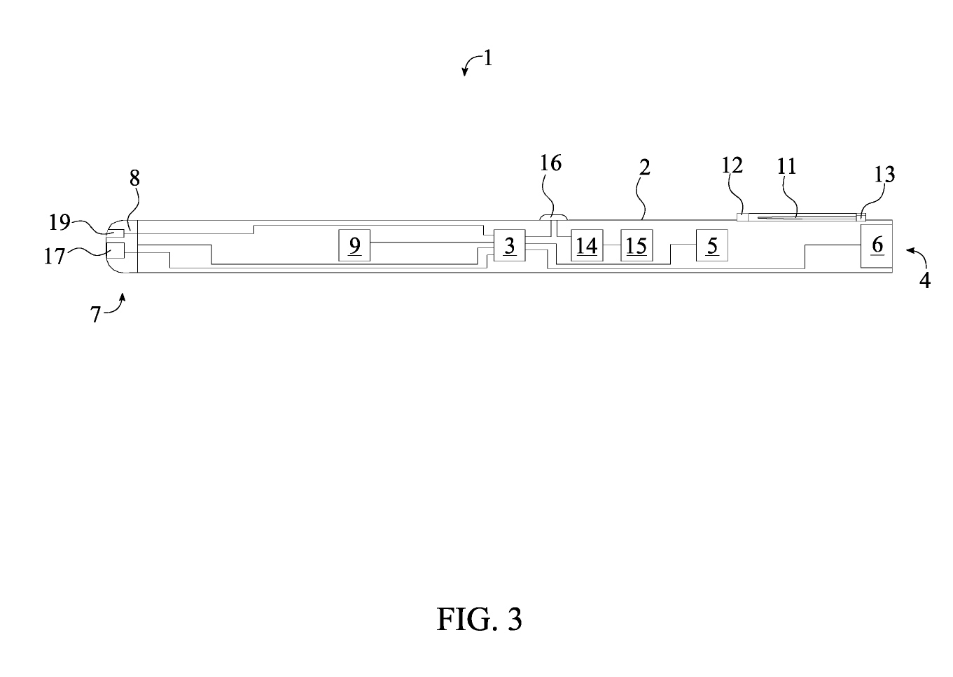

In accordance to FIG. 1 to FIG. 4 and FIG. 6, the present invention comprises a portable health monitoring apparatus 1. As shown in FIG. 3, the portable health monitoring apparatus 1 comprises a pen-shaped housing 2, a central processing unit (CPU) 3, a plurality of health monitoring modules 4, and a first wireless transceiver 5. The pen-shaped housing 2 provides a convenient structure to support and enclose the CPU 3, the plurality of health monitoring modules 4, and the first wireless transceiver 5 within the pen-shaped housing 2. The pen-shaped housing 2 is sized to allow the portable health monitoring apparatus 1 to be easily transported by a user within a pocket of a garment, manipulated by the user, and to house the CPU 3, the plurality of health monitoring modules 4, and the first wireless transceiver 5. The CPU 3 and the first wireless transceiver 5 are mounted within the pen-shaped housing 2. The plurality of health monitoring modules 4 senses the physiological attributes of the user during implementation of the portable health monitoring apparatus 1. The plurality of health monitoring modules 4 is distributed throughout the pen-shaped housing 2. Each of the plurality of health monitoring modules 4 and the first wireless transceiver 5 are electronically connected to the CPU 3. The CPU 3 processes signals received from the plurality of health monitoring modules 4 to be transmitted by the first wireless transceiver 5. The first wireless transceiver 5 transmits the data for a user's physiological attributes to a remote computing device, which is accessed by a professional. The first wireless transceiver 5 is preferred to be a Bluetooth transceiver; however, the first wireless transceiver 5 may be a Wi-Fi, radio frequency, or any other wireless transceiver appropriate to transfer data from one computing device to another.

In some embodiments of the portable health monitoring apparatus 1, the plurality of health monitoring modules 4 comprises an audio input device 6, as shown in FIG. 3. The audio input device 6 receives internal audio sounds from the user's body in order to allow a professional to auscultate for a user, as the professional would with a stethoscope. The audio input device 6 is operatively integrated into the pen-shaped housing 2, wherein the pen-shaped housing 2 is used to press the audio input device 6 against a user's skin in order to record audio from auscultation. The audio input device 6 is positioned opposite to a tip 7 of the pen-shaped housing 2, along the pen-shaped housing 2, such that the audio input device 6 has a sufficient surface area in order to press against the user's skin and record the sounds of auscultation effectively. The audio input device 6 is electronically connected to the first wireless transceiver 5 through the CPU 3 in order to allow the user to transmit an audio file to a remote computing device.

In some embodiments of the portable health monitoring apparatus 1, the plurality of health monitoring modules 4 comprises a temperature transducer 8, as detailed in FIG. 3. The temperature transducer 8 is operatively integrated to the pen-shaped housing 2, wherein the pen-shaped housing 2 is used to press the temperature transducer 8 against a user's skin to assess the user's internal body temperature. The temperature transducer 8 may be placed against the user's ear canal, armpit, or interior of the mouth in order to properly assess the internal body temperature. The temperature transducer 8 is positioned adjacent to the tip 7 of the pen-shaped housing 2 in order to allow the temperature transducer 8 to be easily positioned within a user's ear canal, for example.

In some embodiments of the portable health monitoring apparatus 1, the plurality of health monitoring modules 4 further comprises a glucose meter 9. In accordance to FIG. 1, the glucose meter 9 is laterally integrated into the pen-shaped housing 2. The glucose meter 9 receives a blood test strip to assess the user's concentration of glucose in the user's blood. The glucose meter 9 is especially helpful for users with diabetes such that they can monitor the glucose level in their blood efficiently and conveniently while traveling with the portable health monitoring apparatus 1. In a specific embodiment of the portable health monitoring apparatus 1, the portable health monitoring apparatus 1 further comprises a blood test strip container 10. The blood test strip container 10 is externally and laterally connected to the pen-shaped housing 2. The blood test strip container 10 allows the user to store blood test strips on the pen-shaped housing 2, such that the user is able to conveniently retrieve a blood test strip to test the glucose level in the user's blood. Results from the glucose meter 9 are processed by the CPU 3 to be transmitted to a remote computing device by the first wireless transceiver 5.

Further in a specific embodiment of the portable health monitoring apparatus 1, the portable health monitoring apparatus 1 comprises a needle 11, a needle housing 12, and a spring-loaded release mechanism 13, as shown in FIG. 3. The needle 11 allows the user to puncture the user's skin in order to draw and apply blood onto a blood test strip. The needle housing 12 protects the user from being pricked by the needle 11 unintentionally as the needle 11 is secured within the needle housing 12 when handling the portable health monitoring apparatus 1. The needle housing 12 is externally and laterally mounted to the pen-shaped housing 2, in accordance to FIG. 1 to FIG. 3. The needle housing 12 is positioned opposite to the tip 7 of the pen shaped housing such that the needle housing 12 does not interfere with other health monitoring modules, such as the temperature transducer 8. The spring-loaded release mechanism 13 extends the needle 11 from the needle housing 12 for implementation of the needle 11. The needle 11 is mounted within the needle housing 12 by the spring-loaded release mechanism 13.

In accordance to the preferred embodiment of the portable health monitoring apparatus 1, the portable health monitoring apparatus 1 further comprises a battery 14, as detailed in FIG. 3. The battery 14 provides electrical power to the CPU 3, the plurality of health monitoring modules 4, and the first wireless transceiver 5. The battery 14 is mounted within the pen-shaped housing 2. The battery 14 is electrically connected to the CPU 3, the plurality of health monitoring modules 4, and the first wireless transceiver 5 in order to provide electrical power for operation of the CPU 3, the plurality of health monitoring modules 4, and the first wireless transceiver 5. In a more specific embodiment of the portable health monitoring apparatus 1, the portable health monitoring apparatus 1 further comprises a charging port 15, wherein the battery 14 is rechargeable. In accordance to FIG. 1, the charging port 15 is laterally integrated into the pen-shaped housing 2, in order for the charging port 15 to have a sufficient external interface on the pen-shaped housing 2 for an external power source to be coupled with the charging port 15. The charging port 15 is also laterally integrated along the pen-shaped housing 2 in order to prevent interference with the plurality of health monitoring modules 4, such as the audio input device 6. The charging port 15 is electrically connected to the battery 14, as shown in FIG. 3, such that the charging port 15 allows the battery 14 to be charged after the dissipation from use.

Further in accordance to the preferred embodiment, the portable health monitoring apparatus 1 comprises a power button 16, as detailed in FIG. 1 to FIG. 3. The power button 16 allows the user to toggle the portable health monitoring apparatus 1 on and off. The power button 16 is laterally integrated to the pen-shaped housing 2. The battery 14 is electrically connected to the CPU 3 through the power button 16. This configuration allows the user to interrupt or complete the circuit to turn the portable health monitoring apparatus 1 on or off.

In some embodiments of the portable health monitoring apparatus 1, the portable health apparatus further comprises a camera 17, as detailed in FIG. 1 and FIG. 3. The camera 17 allows the user to record media, such a photographs or videos, for portions of the user's body, such as the interior of the user's ear, nose, throat, or mouth, to be assessed by the professional. The camera 17 is externally integrated to the pen-shaped housing 2. The camera 17 is adjacently positioned to the tip 7 of the pen-shaped housing 2, such that the camera 17 is easily positioned within a user's ear, for example. The camera 17 is electrically connected to the CPU 3 in order to allow the media recorded by the camera 17 to be transmitted through the first wireless receiver 5. In accordance to FIG. 1, a viewing direction 18 of the camera 17 is collinear with the pen-shaped housing 2. The viewing direction 18 is oriented away from the tip 7 of the pen-shaped housing 2 in order to effectively record media within portions of the user's body such as the interior of the user's ear canal.

In some embodiments of the portable health monitoring apparatus 1, the portable health monitoring apparatus 1 comprises a light emission source 19, as shown in FIG. 1 and FIG. 3. The light emission source 19 illuminates the user's ear canal, nasal passage, or throat during implementation in order for the camera 17 to record media with proper exposure. Similar to the camera 17, the light emission source 19 is integrated to the pen-shaped housing 2. The light emission source 19 is adjacently positioned to the tip 7 of the pen-shaped housing 2. In accordance to FIG. 1, an emission direction 20 of the light emission source 19 is collinear with the pen-shaped housing 2. The emission direction 20 is oriented away from the tip 7 of the pen-shaped housing 2. This configuration allows for the illumination of the user's ear canal, nasal passage, or throat to provide proper exposure for recording media with the camera 17 to be transferred to a remote computing device such that a professional is able to assess the media.

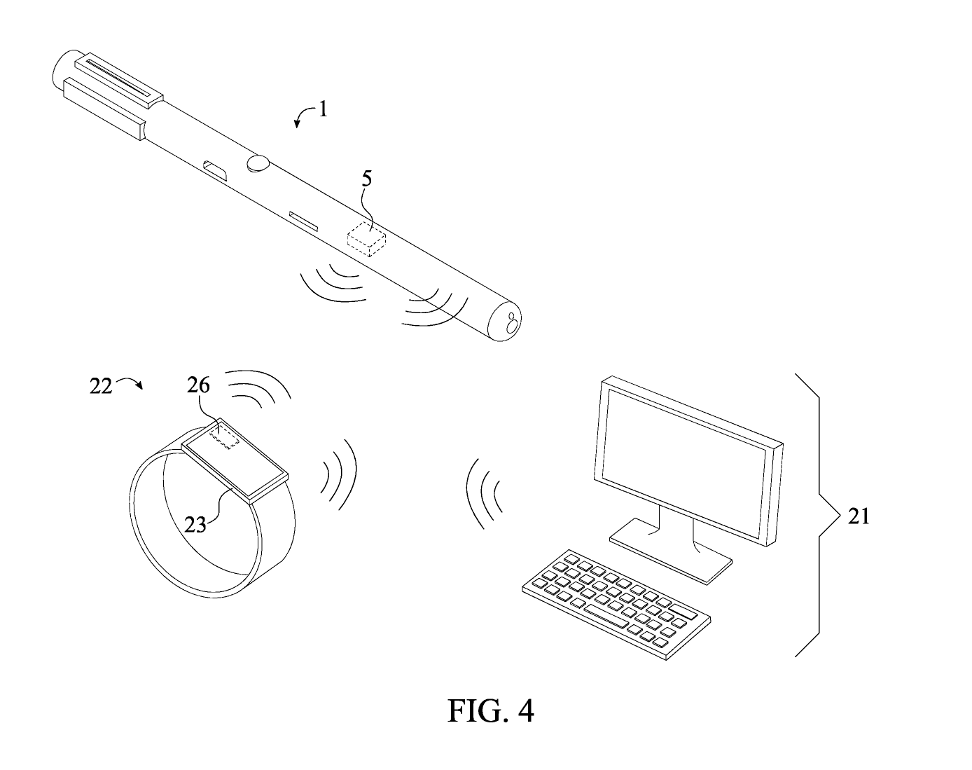

Further in accordance to the preferred embodiment of the present invention, the present invention comprises at least one remote computing device 21, as detailed in FIG. 4. The at least one remote computing device 21 includes, but is not limited to a personal computing device, smartphone, smartwatch, laptop, tablet, or similar computing devices. The first wireless transceiver 5 is communicatively coupled with the at least one remote computing device 21, such that the at least one remote computing device 21 receives data and media from the portable health monitoring apparatus 1 to display to a professional for assessment.

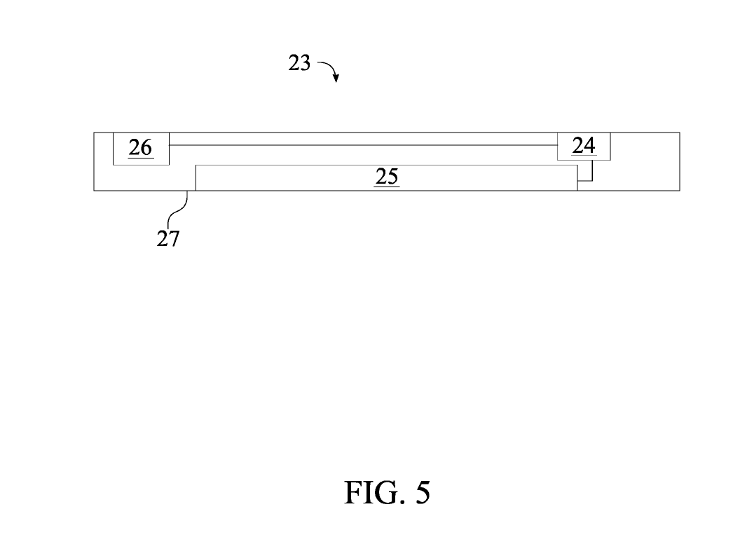

In some embodiments of the present invention, the at least one remote computing device 21 comprises a smartwatch 22, as shown in FIG. 4 and FIG. 5. The smartwatch 22 is worn around the wrist of the user to display or store data acquired from the portable health monitoring apparatus 1, as well as acquire additional data for physiological attributes. In accordance to FIG. 5 the smartwatch 22 comprises a watch case 23, a microprocessor 24, a pressure transducer 25, and a second wireless transceiver 26. The watch case 23 houses the electronic components of the smartwatch 22. The microprocessor 24 processes data from the pressure transducer 25 to be transmitted by the second wireless transceiver 26 as well as process data to be displayed to the user. The pressure transducer 25 monitors the users blood pressure and heart rate. The pressure transducer 25 is positioned adjacent to a wrist-bracing base 27 of the watch in order to interface with the user's wrist when the smart watch is implemented. The second wireless transceiver 26 of the smartwatch 22 is communicatively coupled with the first wireless transceiver 5 in the pen-shaped housing 2 and other remote computing devices from the at least one remote computing device 21, as shown in FIG. 4. The second wireless transceiver 26 transmits data from the pressure transducer 25 to another remote computing device to be assessed by a professional. The second wireless transceiver 26 receives data from the portable health monitoring apparatus 1 to display to the user. The second wireless transceiver 26 is preferred to be a Bluetooth transceiver; however, the second wireless transceiver 26 may be a Wi-Fi, radio frequency, or any other wireless transceiver appropriate to transfer data.

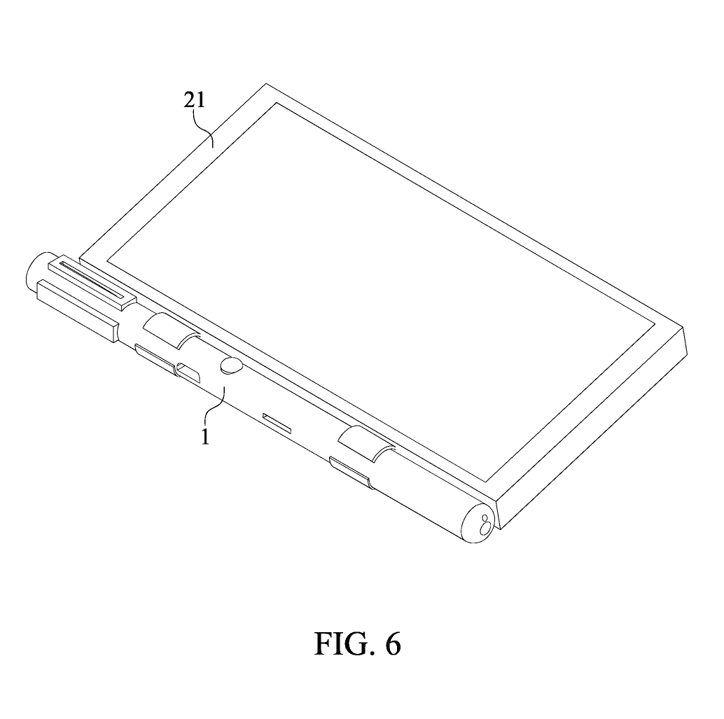

In some embodiments of the present invention, the portable health monitoring apparatus 1 is removably mounted to the at least one remote computing device 21, in accordance to FIG. 6, such that the portable health monitoring apparatus 1 is transported alongside the at least one remote computing device 21. This configuration allows the user convenient access to both the portable health monitoring apparatus 1 and the at least one remote computing device 21 simultaneously, wherein the at least one remote computing device 21 is a smartphone for example. The portable health monitoring apparatus 1 is laterally mounted onto the at least one remote computing device 21 such that the portable health monitoring apparatus 1 does not interfere with operation of the at least one remote computing device 21.

Although the invention has been explained in relation to its preferred embodiment, it is to be understood that many other possible modifications and variations can be made without departing from the spirit and scope of the invention as hereinafter claimed.

What is claimed is:

1. A portable health monitoring system comprises:
a portable health monitoring apparatus;
the portable health monitoring apparatus comprises a pen-shaped housing, a central processing unit (CPU), a plurality of health monitoring modules, and a first wireless transceiver;
the CPU and the first wireless transceiver being mounted within the pen-shaped housing;
the plurality of health monitoring modules being distributed throughout the pen-shaped housing; and
each of the plurality of health monitoring modules and the first wireless receiver being electronically connected to the CPU.

2. The portable health monitoring system, as claimed in claim 1, comprises:
the plurality of health monitoring modules comprises an audio input device;
the audio input device being operatively integrated into the pen-shaped housing, wherein the pen-shaped housing is used to press the audio input device against a user's skin in order to record audio from auscultation;
the audio input device being positioned opposite to a tip of the pen-shaped housing, along the pen-shaped housing; and
the audio input device being electronically connected to the first wireless transceiver through the CPU.

3. The portable health monitoring system, as claimed in claim 1, comprises:
the plurality of health monitoring modules comprises a temperature transducer;
the temperature transducer being operatively integrated to the pen-shaped housing, wherein the pen-shaped housing is used to press the temperature transducer against a user's skin to assess the user's internal body temperature; and
the temperature transducer being positioned adjacent to a tip of the pen-shaped housing.

4. The portable health monitoring system, as claimed in claim 1, comprises:
the plurality of health monitoring modules comprises a glucose meter; and
the glucose meter being laterally integrated into the pen-shaped housing.

5. The portable health monitoring system, as claimed in claim 4, comprises:
the portable health monitoring apparatus further comprises a blood test strip container; and
the blood test strip container being externally and laterally connected to the pen-shaped housing.

6. The portable health monitoring system, as claimed in claim 4, comprises:
the portable health monitoring apparatus further comprises a needle, a needle housing, and a spring-loaded release mechanism;
the needle housing being externally and laterally mounted to the pen-shaped housing;
the needle housing being positioned opposite to a tip of the pen-shaped housing; and
the needle being mounted within the needle housing by the spring-loaded release mechanism.

7. The portable health monitoring system, as claimed in claim 1, comprises:
the portable health monitoring apparatus further comprises a battery;
the battery being mounted within the pen-shaped housing; and
the battery being electrically connected to the CPU, each of the plurality of health monitoring modules, and the first wireless transceiver.

8. The portable health monitoring system, as claimed in claim 7, comprises:
wherein the battery is rechargeable;
the portable health monitoring apparatus further comprises a charging port;
the charging port being laterally integrated into the pen-shaped housing; and
the charging port being electrically connected to the battery.

9. The portable health monitoring system, as claimed in claim 7, comprises:
the portable health monitoring apparatus further comprises a power button;
the power button being laterally integrated to the pen-shaped housing; and
the battery being electrically connected to the CPU through the power button.

10. The portable health monitoring system, as claimed in claim 1, comprises:
the portable health monitoring apparatus further comprises a camera;
the camera being externally integrated to the pen-shaped housing;
the camera being adjacently positioned to a tip of the pen-shaped housing;
the camera being electrically connected to the CPU;
a viewing direction of the camera being collinear with the pen-shaped housing; and
the viewing direction being oriented away from the tip of the pen-shaped housing.

11. The portable health monitoring system, as claimed in claim 1, comprises:
the portable health monitoring apparatus further comprises a light emission source;
the light emission source being integrated to the pen-shaped housing;
the light emission source being adjacently positioned to a tip of the pen-shaped housing;
an emission direction of the light emission source being collinear with the pen-shaped housing; and
the emission direction being oriented away from the tip of the pen-shaped housing.

12. The portable health monitoring system, as claimed in claim 1, comprises:
at least one remote computing device; and
the first wireless transceiver being communicatively coupled with the at least one remote computing device.

13. The portable health monitoring system, as claimed in claim 12, comprises:
the at least one remote computing device comprises a smartwatch;
the smartwatch comprises a watch case, a microprocessor, a pressure transducer and a second wireless transceiver;
the microprocessor, the pressure transducer, and the second wireless transceiver being internally mounted within the watch case;
the pressure transducer being positioned adjacent to a wrist-bracing base of the watch case; and
the second wireless transceiver of the watch being communicatively coupled with the first wireless transceiver in the pen-shaped housing and other remote computing devices from the at least one remote computing device;

14. The portable health monitoring system, as claimed in claim 12, comprises:
the portable health monitoring apparatus being removably mounted to the at least one remote computing device.
